(19) United States
(12) Patent Application Publication
(10) Pub. No.: US 2018/0015080 A1
(43) Pub. Date: Jan. 18, 2018

(54) COMPOSITIONS AND METHODS FOR TREATMENT IN PARKINSON'S DISEASE PATIENTS

(71) Applicant: Ari AZHIR, Los Altos, CA (US)

(72) Inventor: Ari AZHIR, Los Altos, CA (US)

(21) Appl. No.: 15/442,555

(22) Filed: Feb. 24, 2017

Related U.S. Application Data
(63) Continuation of application No. 14/769,907, filed on Aug. 24, 2015, now abandoned.
(60) Provisional application No. 61/769,307, filed on Feb. 26, 2013.

(51) Int. Cl. A61K 31/465; A61K 9/50; A61K 9/24; A61K 9/48; A61K 9/28; A61K 31/198; A61K 9/16

(52) U.S. Cl. CPC A61K 31/465; A61K 31/198; A61K 9/5084; A61K 9/5078; A61K 9/5026; A61K 9/16; A61K 9/4808; A61K 9/28; A61K 9/209; A61K 9/4825

(57) ABSTRACT

The invention provides dosage forms and methods for treating Parkinson's Disease, symptoms resulting from Parkinson's Disease, side effects resulting from treatment of Parkinson's Disease with other pharmaceutical agents, and reducing the progress of Parkinson's Disease. In various embodiments, the dosage forms and methods utilize nicotine and/or salts thereof for once daily administration resulting in four pulsatile releases following administration.

### CROSS-REFERENCE TO RELATED APPLICATIONS

This application claims benefit from U.S. Patent Application No. 61/796,307 (Attorney Docket No. 43297-702.101), filed Feb. 26, 2013, the full disclosure of which is incorporated herein by reference.

### BACKGROUND OF THE INVENTION

Parkinson's Disease is a degenerative disorder of the central nervous system. It results from the death of dopamine-containing cells in a region of the midbrain. The cause of cell death is unknown. The most obvious symptoms are related to movement and balance, including shaking, rigidity, slowness of movement and difficulty with walking and gait. The main motor symptoms are collectively called parkinsonism, or a “parkinsonian syndrome”. The pathology of the disease is characterized by insufficient formation and activity of dopamine produced in certain neurons of parts of the midbrain.

Modern treatments try to manage the early motor symptoms of the disease, mainly through the use of levodopa and dopamine agonists. As the disease progresses and dopamine neurons continue to be lost, a point eventually arrives at which these drugs become ineffective at treating the symptoms and at the same time produce a side effect called dyskinesia, which includes involuntary writhing movements. Therefore, there is a need in the art to treat motor symptoms in subjects with Parkinson's Disease, including symptoms of Parkinson's Disease as well as symptoms indirectly associated with Parkinson's Disease, such as those arising as side effects of treatment. In addition, there is a need in the art for treatments that delay onset or progression of Parkinson's Disease. Moreover, there is a need in the art for simplified treatments to improve compliance with treatment regimens.

### SUMMARY OF THE INVENTION

The present disclosure provides for a pulsatile release dosage form for once-daily administration. In various aspects, the form comprises a capsule or tablet comprising an effective amount of nicotine or salt thereof for treatment of symptoms of Parkinson's Disease or symptoms associated with dopaminergic treatment of Parkinson's Disease or for delay in progression of Parkinson's Disease. In various aspects, the pulsatile release comprises first, second, third, and fourth release peaks, where the first release peak occurs within about two hours of administration to a patient, the second release peak occurs between about five and seven hours of administration to a patient, the third release peak occurs between about eleven and thirteen hours of administration to a patient, and the fourth release peak occurs between about sixteen and twenty hours of administration to a patient. In various aspects, the pulsatile release comprises first, second, third, and fourth release peaks, where said first pulsatile release occurs with a maximum release between about two to four hours of administration, said second pulsatile release occurs with a maximum release between seven and nine hours following administration, said third pulsatile release occurs with a maximum release between thirteen and fifteen hours following administration, and said fourth pulsatile release occurs with a maximum release between eighteen and twenty-two hours following administration. In various aspects, the pulsatile release comprises first, second, third, and fourth release peaks, where said first pulsatile release occurs with a maximum release within two hours of entering the small intestine, said second pulsatile release occurs with a maximum release between seven and nine hours following administration, said third pulsatile release occurs with a maximum release between thirteen and fifteen hours following administration, and said fourth pulsatile release occurs with a maximum release between eighteen and twenty-two hours following administration.

In various embodiments, the present disclosure provides for an oral pulsatile release dosage form for once-daily oral administration, said form comprising an effective amount of nicotine or a salt thereof for treatment of symptoms of Parkinson's Disease or symptoms associated with dopaminergic treatment of Parkinson's Disease or for delay in progression of Parkinson's Disease, wherein said form exhibits first, second, third, and fourth pulsatile releases of nicotine or salt thereof, wherein said first pulsatile release occurs with a maximum release within two hours of administration, said second pulsatile release occurs with a maximum release between five and seven hours following administration, said third pulsatile release occurs with a maximum release between eleven and thirteen hours following administration, and said fourth pulsatile release occurs with a maximum release between sixteen and twenty hours following administration. In various embodiments, said first pulsatile release occurs with a maximum release within one hour of administration, said second pulsatile release occurs with a maximum release at about six hours following administration, said third pulsatile release occurs with a maximum release at about twelve hours following administration, and said fourth pulsatile release occurs with a maximum release at about eighteen hours following administration.

In various embodiments, pulsatile release provides for at least 80% of the total release of nicotine or salt thereof from the respective pulsatile release as occurring within one hour of the time of maximum release for the pulsatile release. In one embodiment, the amount is at least 90%. In various embodiments, less than 10% of the total amount of nicotine or salt thereof in the dosage form is released outside of one of the pulsatile releases. For example, one embodiment, less than 10% of the total amount of nicotine or salt thereof is released outside of one hour from the time of a pulsatile release peak.

In various embodiments, each pulsatile release comprises from about 0.1 to 10 mg nicotine or salt thereof for a total amount of nicotine or salt thereof in the dosage form of from about 0.4 to 40 mg. In various embodiments, each pulsatile release independently comprises an amount of nicotine or salt thereof selected from about 1 mg, about 2 mg, about 3 mg, about 4 mg, about 5 mg, and about 6 mg. Alternatively, each pulsatile release includes about the same amount of nicotine or salt thereof. For example, in various embodiments, each pulsatile release comprises an amount of nicotine or salt thereof selected from about 1 mg for a combined total amount in the dosage form of about 4 mg, about 2 mg for a combined total amount in the dosage form of about 8 mg, about 3 mg for a combined total amount in the dosage form of about 12 mg, about 4 mg for a combined total amount in the dosage form of about 16 mg, about 5 mg for a combined total amount in the dosage form of about 20 mg, and about 6 mg for a combined total amount in the dosage form of about 24 mg.

In various embodiments, the dosage form comprises an effective amount of nicotine bitartrate dihydrate. In various embodiments, the dosage form comprises an effective amount of nicotine (free base). In various embodiments, the dosage form comprises nicotine polacrilex. In various embodiments, the dosage form comprises a mixture of the above, or alternatively, a mixture of nicotine salts. In various embodiments, the dosage form comprises an effective amount of one or more nicotine salts, and nicotine (free base) is excluded, or is present in less than an effective amount, or is present in a trace or de minimus amount.

In various embodiments, the dosage form is a capsule. For example, in various embodiments, a capsule according to the present disclosure comprises a liquid, powder, polymeric matrix, or coating layer comprising nicotine or salt thereof for providing said first pulsatile release upon administration to a patient; and said capsule further comprises beads comprising nicotine or salt thereof for providing said second, third, and fourth pulsatile releases upon administration to a patient. In various embodiments, beads are selected from the group consisting of enteric-coated beads, erodible-matrix beads, wax-coated beads, ethylcellulose-coated beads, silicone elastomer coated beads, and combinations thereof. In various embodiments, the capsule comprises a water-swellable polymeric membrane, which may rupture following administration to a patient. In various embodiments, the capsule comprises a hard gelatin outer surface.

In various embodiments, the dosage form is a tablet. For example, in various embodiments, the tablet comprises an immediate release coating and a core, wherein said immediate release coating comprises nicotine or salt thereof for said first pulsatile release, and said core comprises nicotine or salt thereof for said second, third, and fourth pulsatile releases. In various embodiments, the immediate release coating is selected from an erodible-matrix coating, a wax coating, an ethylcellulose coating, a silicone elastomer coating, and combinations thereof.

In various embodiments, the dosage form also comprises levodopa, carbidopa, or a combination thereof. In various embodiments, a dopaminergic agent is excluded. For example, in various embodiments, the dosage form and/or method of treatment does not include levodopa and/or carbidopa.

In various embodiments, the dosage form is capable of being administered so that one or more metabolites of said nicotine or salt thereof achieves a plasma level of about 1 to about 500 ng/ml within one hour of each of said pulsatile releases.

The present disclosure also provides methods for treatment of symptoms of Parkinson's Disease or symptoms associated with dopaminergic treatment of Parkinson's Disease or for delay in progression of Parkinson's Disease. In various embodiments, the methods comprise administering a dosage form as described herein to a patient in need thereof. In various embodiments, the treated symptoms of Parkinson's Disease are gait and balance problems. In various embodiments, the treated symptoms are associated with dopaminergic treatment of Parkinson's Disease are levodopa-induced dyskinesias. In various embodiments, the treatment is for a delay in progression of Parkinson's Disease or for a reduction in the incidence of Parkison's Disease in patients at risk of developing Parkinson's Disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

As used herein, the term “pH independent release” refers to a rate of release of a drug from a dosage form that does not change when the pH of the environment in which the dosage form is found is changed, e.g., from an acidic pH to a higher pH. The term “pH dependent release” refers to a rate of release of a drug from a dosage form that changes when the pH of the environment in which the dosage form is found is changed, for example from a low pH to a higher pH.

An “average” as used herein is preferably calculated in a set of normal human subjects, this set being at least about 3 human subjects, preferably at least about 5 human subjects, preferably at least about 10 human subjects, even more preferably at least about 25 human subjects, and most preferably at least about 50 human subjects.

The term “animal” or “animal subject” as used herein includes humans as well as other mammals. The methods generally involve the administration of one or more drugs for the treatment of one or more diseases. Combinations of agents can be used to treat one disease or multiple diseases or to modulate the side-effects of one or more agents in the combination.

As used herein, the term “zero-order release” refers to a uniform or nearly uniform rate of release of a drug from a dosage form during a given period of release, a rate of release that is independent of the concentration of drug in the dosage form. A dosage form with a zero-order release profile is referred to herein as a “zero-order dosage form.” In various embodiments, dosage forms disclosed herein display release profiles that are not zero-order release forms.

The term “oral administration,” as used herein, refers a form of delivery of a dosage form of a drug to a subject, wherein the dosage form is placed in the mouth of the subject and swallowed.

The term “orally deliverable” herein means suitable for oral administration.

The term “enteric coating,” as used herein, refers to a tablet coating that is resistant to gastric juice, and which dissolves after a dosage form with the enteric coating passes out of the stomach, after oral administration to a subject.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any combination thereof.

The term “excipient,” as used herein, means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, lubricants, glidants, substances added to mask or counteract a disagreeable taste or odor, flavors, dyes, fragrances, and substances added to improve appearance of the composition.

“Pharmaceutically acceptable salt” refers to salts of drug compounds derived from the combination of such compounds and an organic or inorganic acid (acid addition salts) or an organic or inorganic base (base addition salts). The agents, including drugs, contemplated for use herein may be used in either the free base or salt forms, with both forms being considered as being within the scope of the certain present invention embodiments. Where a given salt form of nicotine is too insoluble to provide desired pulsatile release characteristics using a dosage form of the present invention, it may be preferred to use a more soluble salt in the dosage form.

The terms “tablet core,” “matrix,” and “tablet core matrix” refer to a compressed tablet prior to coating. No specialized geometry of the tablet core is necessary in the present invention. The tablet core may be in any shape known in the pharmaceutical industry and suitable for drug delivery, such as in spherical, cylindrical, or conical shape. In the case of cylindrical shape, it generally has flat, convex, or concave surfaces.

“Substantially eliminated” as used herein encompasses no measurable or no statistically significant effect (such as with one or more side effects), when nicotine or salt thereof is administered.

The term “treating” and its grammatical equivalents as used herein include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

### Pulsatile Release Profile

In various embodiments, the release profile is a pulsatile release. For example, in various embodiments, an immediate release of nicotine is followed by additional pulsatile releases of nicotine or salt thereof. In various embodiments, the first pulsatile release is time-delayed to allow for progression through the stomach into the small intestine, for example by about two hours, and the additional pulsatile releases are timed accordingly to be distributed throughout a 24 hour time period. In various embodiments, the first pulsatile release is delayed such that release occurs through a pH-dependent mechanism to allow for progression through the stomach into the small intestine, and the additional pulsatile releases are delayed accordingly to be distributed throughout a 24 hour time period.

In various embodiments, nicotine or salt thereof is present about 10 mg or less per pulsatile release, or present at about 6 mg or less per pulsatile release, or present at about 4 mg or less per pulsatile release, or present at about 3 mg per pulsatile release. In various embodiments, a first pulse of about 1-2 mg or less nicotine or salt thereof is released in a first release, and a second, third, and fourth pulses of about 2-3 mg or less nicotine or salt thereof for each pulse is released.

In various embodiments, the dosage form is capable of being administered so that one or more metabolites of said nicotine or salt thereof achieves a plasma level of about 1 to about 500 ng/ml within an hour of each pulsatile release.

A pH-dependent delayed release characteristic of one embodiment of the dosage form of the present disclosure result from an enteric coating. Once the dosage form leaves the highly acidic environment of the stomach and enters the higher pH of the lower gastrointestinal tract, the enteric coating dissolves, and the tablet core matrix controls the rate of release of drug remaining therein. The enteric coating preferably dissolves at a pH of at least about 5. In some embodiments, the enteric coating dissolves at a pH of at least about 5.5, 6.0, 6.5, 7.0, 7.5, or 8.0.

In some embodiments, the dosage form comprises an enteric coating and nicotine or salt thereof, wherein the enteric coating dispenses the nicotine in a metered fashion when said pH is above about 5.0. In some embodiments, the dosage form comprises an enteric coating and nicotine or salt thereof, wherein the enteric coating dispenses the nicotine in a metered fashion when said pH is above about 5.5, 6.0, 6.5, 7.0, 7.5, or 8.0.

In some embodiments, in addition to a pH-dependent release rate, the dosage form of the present invention described has a controlled release rate, e.g., a zero-order release rate through changes in pH, such as occur when the dosage form passes from the stomach to the upper intestines of a subject after oral administration thereto. In the case of a human being the average pH of the fluids in a stomach is about pH 1, while the average pH of the upper intestinal tract is about pH 5 to about 7. In various embodiments, dosage forms disclosed herein display release profiles that are not zero-order release forms.

In some embodiments, an enteric coating is combined with a pore former to effect a pH-independent extended release. A pore former can allow a limited amount of environmental fluids to reach the tablet core in the upper gastrointestinal (GI) tract, including the stomach, thereby permitting a limited amount of drug to be released into the subject at that stage after oral administration. In embodiments containing pore forming agents, the drug in the tablet core diffuses out of the tablet and into the environment surrounding the tablet through channels formed initially through pore forming agents in the enteric coating, and later, after the enteric coating has dissolved, through channels formed in the matrix itself.

In some cases, an enteric coating can be used to increase the burst effect associated with matrix tablets. This effect is thought to be related to the size of the surface area of a tablet, and to be caused by the amount of drug located on or near the surface of the tablet. This effect can be increased through the coating of a tablet core matrix with an enteric coating with multiple pore-formers distributed therein, as described above. For this embodiment of the invention, the solubility of the drug in the tablet core need be pH dependent. It is contemplated that any drug could be used in this embodiment of the invention, provided its solubility characteristics allow for containment within and release from the matrix. The enteric coating with pore formers effectively minimizes the surface area of the tablet that is initially exposed to solution in the GI tract and thus limits the amount of drug that is initially released. The coating composition, in terms of ratio of enteric to pore-former, could be changed to dictate how much the burst is maximized and therefore the release rate of the drug. A pH-sensitive enteric coating dissolves when the tablet enters the intestine and allows the core to take over the control of the tablet release.

The dosage form of the present invention can delay the period of drug release compared to uncoated tablet cores having the same composition as the tablet cores of the present dosage forms. The drug in the coated tablet cores of the present invention delay release of the drug into a subject by at least about 30 minutes, 1 hour, 1.5 hours, 2 hours, 2.5 hours, 3 hours, 4 hours, 5 hours, 6 hours, 7 hours, or 8 hours after oral administration. A dosage form that provides delayed drug release as described herein can be formulated for once-daily administration with multiple pulsatile releases.

The dosage form of the present invention can extend the period of drug release compared to uncoated tablet cores having the same composition as the tablet cores of the present dosage forms. The drug in the coated tablet cores of the present invention preferably continue to release pulses or bursts of the drug into a subject to at least 10 hours, more preferably to at least 12 hours, even more preferably to at least 14 hours, and most preferably to at least 16 hours after oral administration. A dosage form that provides pulsatile or burst drug release over about 10, 11, 12, 13, or 14 hours or more can be formulated for once daily administration, thereby allowing pulsatile delivery of a drug over a 24-hour period.

### Pharmaceutical Compositions

Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

### Tablets and Capsules

In some aspects, the present disclosure provides for a pulsatile release dosage form for once-daily administration. In various embodiments, the form comprises a capsule or tablet comprising an effective amount of nicotine for treatment of symptoms of Parkinson's Disease or symptoms associated with dopaminergic treatment of Parkinson's Disease, wherein said capsule or tablet exhibits pulsatile release of said nicotine over the course of a day. In various aspects, said pulsatile release comprises a first, second, third, and fourth release peak, wherein said first release peak occurs within about two hours of administration to a patient, and said second release peak occurs between about five and seven hours of administration to a patient, said third release peak occurs between about eleven and thirteen hours of administration to a patient, and said fourth release peak occurs between about sixteen and twenty hours of administration to a patient. In various aspects, said capsule or tablet achieves an efficacious peak plasma concentration of nicotine or a metabolite thereof within one hour from each pulsatile peak.

Dosage forms according to the invention include capsules and tablets. In various embodiments, said capsule comprises a powder comprising nicotine for providing said first release peak upon administration to a patient, and said capsule further comprises beads comprising nicotine for providing said second, third, and fourth release peaks upon administration to a patient. Beads are selected from the group consisting of enteric-coated beads, erodible-matrix beads, wax-coated beads, ethylcellulose-coated beads, silicone elastomer coated beads, and combinations thereof. In various embodiments, said capsule comprises a water-swellable matrix to provide a gastroretentive formulation with repeated pulsatile release in the stomach. A water-swellable matrix may comprise polyethylene oxide, hydroxypropylmethylcellulose, and combinations thereof.

In various embodiments, the dosage form comprises a water-swellable polymeric membrane. Preferably, the water-swellable polymeric membrane ruptures following administration to a patient.

In various embodiments, the dosage form is a tablet. Tablets comprising at least one coating and a core, wherein an outermost coating comprises nicotine for the first release peak, and said core comprises nicotine for the second, third, and fourth release peaks, are encompassed. In various embodiments, the coating is selected from an enteric coating, an erodible-matrix coating, a wax coating, an ethylcellulose coating, a silicone elastomer coating, and combinations thereof.

In some embodiments, the invention includes a multilayer tablet comprising an immediate release layer and pulsatile release layer(s). In some embodiments, the immediate release layer comprises nicotine or a metabolite. In some embodiments, each pulsatile release layer comprises nicotine or a metabolite. In some embodiments, the immediate release layer and each pulsatile release layer comprise nicotine or a metabolite.

The tablet core of the dosage form of the present invention can comprise a matrix of a drug and a water soluble polymer, suitable for pulsatile release upon entry and following exit of the tablet from the acidic environment of the stomach and dissolution of the coating upon entry into the higher pH environment of the intestine.

The tablet core is prepared by conventional dry granulation methods without using a solvent. The enteric coating is applied using a conventional process known in the art. The coated tablets of the present invention have a dual advantage in allowing ease of manufacture and affording medicament release in a substantially pulsatile fashion over an extended period of time.

In some embodiments, the dosage form comprises an enteric coating comprising an enteric polymer. Suitable enteric polymers include, but are not limited to, methacrylic acid/methacrylic acid ester copolymer, a methacrylic acid/acrylic acid ester copolymer, cellulose acetate phthalate, hydroxypropyl methylcellulose pthalate, hydroxypropyl methyl cellulose acetate succinate, cellulose acetate trimellitate, and polyvinyl acetate phthalate.

Enteric polymers suitable for use in the present invention include, but are not limited to polyacrylate copolymers such as methacrylic acid/methacrylic acid ester copolymers or methacrylic acid/acrylic acid ester copolymers, such as USP/NF, Types A, B, or C, which are available from Rohm GmbH under the brand name Eudragit™; cellulose derivatives, such as cellulose acetate phthalate, hydroxypropyl methylcellulose pthalate, hydroxypropyl methyl cellulose acetate succinate, and cellulose acetate trimellitate; and polyvinyl acetate phthalate, such as is available from Colorcon, under the brand name SURETERIC®, and the like. In some embodiments, the enteric polymer is a polyvinyl acetate phtalate.

Suitable water soluble pore-forming agents for use in the enteric coating in the dosage forms of the present invention include, but are not limited to, povidone K 30, polyvinyl alcohol, cellulose derivatives such as hydroxypropyl cellulose, hydroxypropyl methyl cellulose, methyl cellulose or sodium carboxymethylcellulose; sucrose; xylitol, sorbitol, mannitol, maltose, xylose, glucose, potassium chloride, sodium chloride, polysorbate 80, polyethylene glycol, propylene glycol, sodium citrate, or combinations of any of the above. The pore-forming agent preferably comprises hydroxypropyl methyl cellulose.

The composition of the enteric coating is preferably designed to ensure adherence of the coating to the tablet core. Methods for selection of coating compositions that adhere to compressed tablets are known. See, for example, Pharmaceutical Dosage Forms: Tablets, 2nd ed., vol. 1, Lieberman et al., ed. (Marcel Dekker, Inc.; New York, N.Y.; 1989), pp. 266-271, incorporated herein by reference. Additionally, the cores can be subcoated prior to coating with an enteric coating. The subcoat can function; to provide that pores in the core are filled in prior to coating with an enteric coat to insure against coating failure. The sub-coat can consist of any film forming formulation examples include Opadry (Colorcon), Opadry II (Colorcon), AMT (Colorcon) and HPMC.

The enteric coating can be about 3% to about 10% by weight of the dosage form of the present invention. In some cases, the enteric coating can be about 4%, 5%, 6%, 7%, 8%, 9%, 10%, 11%, 12%, 13%, 14%, 15%, 16%, 17%, 18%, 19%, or 20% by weight of the dosage form of the present invention.

In some embodiments, the tablet core of a dosage form of the invention comprises at least one hydrophilic polymer. Suitable hydrophilic polymers include, but are not limited to, hydroxypropyl methylcellulose (hereinafter, “HPMC”), hydroxypropylcellulose, or other water soluble or swellable polymers such as sodium carboxymethyl cellulose, xanthan gum, acacia, tragacanth gum, guar gum, karaya gum, alginates, gelatin, and albumin. The hydrophilic polymers can be present in amounts ranging from about 5% to about 95% by weight of the system. In some embodiments, the hydrophilic polymers are selected from the group consisting of cellulose ethers, such as hydroxypropylmethylcellulose, hydroxypropylcellulose, methylcellulose, and mixtures thereof.

Pharmaceutical Components

Dosage forms of the present invention may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Diluents can be incorporated into the tablet core of a dosage form.

Dosage forms of the invention, preferably a tablet core matrix, optionally comprise one or more pharmaceutically acceptable diluents as excipients. Non-limiting examples of suitable diluents include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of amorphous cellulose (e.g., Rexcel™) and powdered cellulose; calcium carbonate; glycine; bentonite; polyvinylpyrrolidone; and the like. Such diluents, if present, constitute in total about 5% to about 99%.

In another embodiment of the invention, a gastric retained dosage form of nicotine or salt thereof is provided. Exemplary polymers include polyethylene oxides, alkyl substituted cellulose materials and combinations thereof, for example, high molecular weight polyethylene oxides and high molecular weight or viscosity hydroxypropylmethylcellulose materials. Further details regarding an example of this type of dosage form can be found in Shell, et al., U.S. Pat. No. 5,972,389 and Shell, et al., WO 9855107, and U.S. Pat. No. 8,192,756, the contents of each of which are incorporated by reference in their entirety.

In yet another embodiment, a bi, tri, or quad-layer tablet releases nicotine or salt thereof to the upper gastrointestinal tract from an active containing layer, while the other layer is a swelling or floating layer. Details of this dosage may be found in Franz, et al., U.S. Pat. No. 5,232,704. This dosage form may be surrounded by a band of insoluble material as described by Wong, et al., U.S. Pat. No. 6,120,803.

In some embodiments, nicotine is orally administered using an orally disintegrating tablet. Examples of orally disintegrating tablets are known, such as disclosed in U.S. Pat. Nos. 7,282,217; 7,229,641; 6,368,625; 6,365,182; 6,221,392; and 6,024,981.

Another embodiment of the invention uses a gastric retained swellable tablet having a matrix comprised of polyethylene oxide and hydroxypropylmethylcellulose. Further details may be found in Gusler, et al. “Optimal Polymer Mixtures for Gastric Retentive Tablets,” granted as U.S. Pat. No. 6,723,340, the disclosure of which is incorporated herein by reference.

Pharmaceutically acceptable carriers for therapeutic use are well known in the pharmaceutical art, and are described, for example, in Remington's Pharmaceutical Sciences, Mack Publishing Co. (A. R. Gennaro edit., 1985). Preservatives, stabilizers, dyes and other ancillary agents may be provided in the pharmaceutical composition. For example, sodium benzoate, sorbic acid and esters of p-hydroxybenzoic acid may be added as preservatives. In addition, antioxidants and suspending agents may be used.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose, microcrystalline cellulose, and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; hydroxypropylmethylcellulose; hydroxypropylcellulose (e.g., Klucel™); and ethylcellulose (e.g., Ethocel™). Such binding agents and/or adhesives, if present, can constitute in total about 0.5% to about 25%, about 0.75% to about 15%, or about 1% to about 10%, of the total weight of the composition.

Compositions of the invention optionally comprise one or more pharmaceutically acceptable lubricants (including anti-adherents and/or glidants) as excipients. Suitable lubricants include, either individually or in combination, glyceryl behenate (e.g., Compritol™ 888); stearic acid and salts thereof, including magnesium, calcium and sodium stearates; hydrogenated vegetable oils (e.g., Sterolex™); colloidal silica; talc; waxes; boric acid; sodium benzoate; sodium acetate; sodium fumarate; sodium chloride; DLleucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition. In some embodiments, magnesium stearate is a lubricant used to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition. Other excipients such as colorants, flavors and sweeteners are known in the pharmaceutical art and can be used in compositions of the present invention.

In some embodiments, the dosage form of the present disclosure comprises: a tablet core comprising nicotine bitartrate dehydrate, magnesium stearate, and microcrystalline cellulose. In some embodiments, the dosage form comprises: a tablet core comprising nicotine in a water soluble polymer matrix; and an enteric coating comprising an enteric polymer and, optionally, a pore-former; wherein, the tablet core or the enteric coating or both include at least one excipient. The dosage form comprises at least one excipient preferably selected from the group consisting of pharmaceutically acceptable diluents, binding agents and lubricants. In some cases, a dosage form comprises at least one excipient selected from the group consisting of lactose (e.g., lactose monohydrate), polyvinylpyrrolidone, magnesium stearate and microcrystalline cellulose. In the above embodiments, additional components are provided such that the dosage form is capable of multiple pulsatile releases of the nicotine or salt thereof.

Standard methods of production are suitably used to produce the dosage forms of the present invention. Dry mixing of intragranular ingredients, followed by granulation, and dry mixing of intragranular ingredients with extragranular ingredients are standard techniques used in the industry. See, for example, Chapter 4 (“Compressed Tablets by Direct Compression,” by Ralph F. Shangraw) of Pharmaceutical Dosage Forms: Tablets, vol. 1, 2 ed., Lieberman et al. ed., Marcel Dekker, Inc. pub. (1989), pp. 195-246. The enteric coating is suitably applied using any standard coating technique, such as the techniques described in Chapter 5 (“Compression-Coated and Layer Tablets”, by William C. Gunsel et al.), of the same volume.

### Nicotine

Nicotine may be isolated and purified from nature or synthetically produced in any manner. The term “nicotine” is also intended to encompass the commonly occurring salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate, ethanesulfonate, benzenesulfonate, p-toluene sulfonate, camphorate and pamoate salts. Nicotine is a colorless to pale yellow, strongly alkaline, oily, volatile, hygroscopic liquid having a molecular weight of 162.23. The systematic name of nicotine is 3-[(2S)-1-methylpyrrolidin-2-yl]pyridine and its structure is:

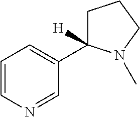

Unless specifically indicated otherwise, the term “nicotine” further includes any pharmacologically acceptable derivative or metabolite of nicotine which exhibits pharmacotherapeutic properties similar to nicotine. Such derivatives, metabolites, and derivatives of metabolites are known in the art, and include, but are not necessarily limited to, cotinine, norcotinine, nornicotine, nicotine N-oxide, cotinine N-oxide, 3-hydroxycotinine and 5-hydroxycotinine or pharmaceutically acceptable salts thereof. A number of useful derivatives of nicotine are disclosed within the Physician's Desk Reference (most recent edition) as well as Harrison's Principles of Internal Medicine. Methods for production of nicotine derivatives and analogues are well known in the art. See, e.g., U.S. Pat. Nos. 4,590,278; 4,321,387; 4,452,984; 4,442,292; and 4,332,945.

In various embodiments, the dosage form comprises an effective amount of nicotine bitartrate dihydrate. In various embodiments, the dosage form comprises an effective amount of nicotine (free base). In various embodiments, the dosage form comprises nicotine polacrilex.

### Dopaminergic Agents

In one aspect, the invention provides compositions and methods to reduce or eliminate the effects of a dopaminergic agent. In some embodiments, the compositions and methods retain or enhance a desired effect of the dopaminergic agent, e.g., antiparkinsonian effect. The methods and compositions of the invention apply to any dopaminergic agent for which it is desired to reduce one or more side effects. In some embodiments, the compositions and methods of the invention utilize a dopamine precursor. In some embodiments, the compositions and methods of the invention utilize a dopamine agonist. In some embodiments, the dopaminergic agent is levodopa, bromocriptine, pergolide, pramipexole, cabergoline, ropinorole, apomorphine or a combination thereof. In some embodiments, the dopaminergic agent is levodopa. In some embodiments, the compositions and methods of the invention utilize one or more agents used in the art in combination with a dopamine agent treatment to achieve a therapeutic effect. For instance, in one exemplary embodiment the compositions and methods of the invention utilize levodopa in combination with an agent such as carbidopa, which blocks the conversion of levodopa to dopamine in the blood. In another exemplary embodiment, the compositions and methods of the invention utilize levodopa in combination with a COMT Inhibitor, such as entacapone. In another exemplary embodiment, the compositions and methods of the invention utilize levodopa in combination with a monoamine oxidase type B (MAO-B) inhibitor such as selegiline. In yet another exemplary embodiment, the compositions and methods of the invention utilize levodopa in combination with amantadine.

Levodopa, an aromatic amino acid, is a white, crystalline compound, slightly soluble in water, with a molecular weight of 197.2. It is designated L-3,4-dihydroxyphenylalanine (S)-2-amino-3-(3,4-dihydroxyphenyl)propanoic acid. Its structural formula is

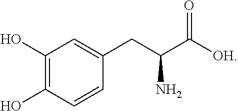 [SMILES: N[C@@H](Cc1ccc(O)c(O)c1)C(=O)O]

Levodopa is used for the treatment of Parkinson's disease. However, although initially very effective, longterm treatment with levodopa gives rise to multiple complications. The principal adverse reactions of dopaminergic agent include headache, diarrhea, hypertension, nausea, vomiting, involuntary movements (e.g. dyskinesias), mental disturbances, depression, syncope, hallucinations, and abnormal renal function.

The invention provides compositions and methods utilizing nicotine or salt thereof that reduces or eliminates a side effect associated with dopaminergic agent treatment.

### Methods of Treatment

In some embodiments, the invention provides compositions and methods utilizing nicotine or salt thereof to reduce, alleviate, or eliminate symptoms of Parkinson's Disease or symptoms associated with Parkinson's Disease, e.g., a side effect associated with dopaminergic agent treatment. In some embodiments, the invention provides compositions and methods utilizing nicotine, e.g., to reduce or eliminate a side effect associated with dopaminergic agent treatment. In some embodiments, the nicotine reduces or eliminates a side effect associated with dopaminergic agent treatment. Dopaminergic agents include a dopamine precursor or a dopamine receptor agonist. Examples of dopaminergic agents include levodopa, bromocriptine, pergolide, pramipexole, cabergoline, ropinorole, apomorphine or a combination thereof.

In some embodiments the invention provides methods of decreasing a side effect of a dopaminergic agent in an animal, e.g. a human, that has received an amount of the dopaminergic agent sufficient to produce a side effect by administering to the animal, e.g., human, an amount of nicotine or salt thereof in a release profile sufficient to reduce or eliminate the side effect.

The side effect may be acute or chronic. The effect may be biochemical, cellular, at the tissue level, at the organ level, at the multi-organ level, or at the level of the entire organism. The effect may manifest in one or more objective or subjective manners, any of which may be used to measure the effect. If an effect is measured objectively or subjectively (e.g., dyskinesias and the like), any suitable method for evaluation of objective or subjective effect may be used. Examples include visual and numeric scales and the like for evaluation by an individual. A further example includes sleep latency for measurement of drowsiness, or standard tests for measurement of concentration, mentation, memory, and the like. These and other methods of objective and subjective evaluation of side effects by an objective observer, the individual, or both, are known in the art.

In some embodiments, the invention provides a composition comprising nicotine (or salt thereof), wherein the nicotine is present in an amount sufficient and released with pharmacokinetics sufficient to decrease a side effect of a dopaminergic agent by a measurable amount, compared to the side effect without the nicotine, when the composition is administered to an animal. In some embodiments, a side effect of the dopaminergic agent is decreased by an average of at least about 5, 10, 15, 20, 25, 30, 35, 40, 45, 50, 55, 60, 65, 70, 75, 80, 85, 90, 95, or more than 95%, compared to the side effect without the nicotine. In some embodiments, a side effect of the dopaminergic agent is decreased by an average of at least about 5%, compared to the side effect without the nicotine. In some embodiments, a side effect of the dopaminergic agent is decreased by an average of at least about 10%, compared to the side effect without the nicotine. In some embodiments, a side effect of the dopaminergic agent is decreased by an average of at least about 15%, compared to the side effect without the nicotine. In some embodiments, a side effect of the dopaminergic agent is decreased by an average of at least about 20%, compared to the side effect without the nicotine. In some embodiments, a side effect of the dopaminergic agent is decreased by an average of at least about 30%, compared to the side effect without the nicotine. In some embodiments, a side effect is substantially eliminated compared to the side effect without the nicotine. In some embodiments, the side effect is dyskinesias.

In various embodiments, the methods of treatment are directed to direct symptoms of Parkinson's Disease rather than treatment of a dopaminergic agent-induced side effect. For example, in various embodiments, the methods of treatment are directed to treatment of gait and balance deficits resulting directly from Parkinson's Disease. In various embodiments, nicotine is administered separately from any dopaminergic agent. In various embodiments, the subject undergoing treatment with nicotine is not receiving a dopaminergic agent. In various embodiments, the subject undergoing treatment with nicotine is not receiving levodopa, carbidopa, or combinations thereof.

In various embodiments, the methods are directed to slowing the progression of Parkison's Disease, or to prophylaxis of Parkinson's Disease, or to reducing the incidence of Parkinson's Disease in those at risk of developing Parkinson's Disease.

### Dosing and Administration

In some embodiments, the daily dose of nicotine is between about 0.4 and 40 mg per day oral administration. In some embodiments, the daily dose of nicotine is about, less than about or more than about 0.9 mg. In some embodiments, the daily dose of nicotine is about, less than about or more than about 1.8 mg. In some embodiments, the daily dose of nicotine is about, less than about or more than about 2.4 mg. In some embodiments, the daily dose of nicotine is about, less than about or more than about 3 mg. In some embodiments, the daily dose of nicotine is about, less than about or more than about 6 mg. In some embodiments, the daily dose of nicotine is about, less than about or more than about 7 mg. In some embodiments, the daily dose of nicotine is about, less than about or more than about 8 mg. In some embodiments, the daily dose of nicotine is about, less than about or more than about 9 mg. In some embodiments, the daily dose of nicotine is about, less than about or more than about 12 mg. In some embodiments, the daily dose of nicotine is about, less than about or more than about 14 mg. In some embodiments, the daily dose of nicotine is about, less than about or more than about 18 mg. In some embodiments, the daily dose of nicotine is about, less than about or more than about 21 mg. In some embodiments, the daily dose of nicotine is about, less than about or more than about 24 mg. In some embodiments, the daily dose of nicotine is about, less than about or more than about 32 mg.

In some embodiments, an effective amount of nicotine is administered such that the nicotine or a metabolite of the nicotine reaches a critical concentration in the bloodstream, plasma, or the tissue. In some embodiments, the nicotine is administered such that the nicotine or a metabolite of nicotine reaches a critical peak concentration in the bloodstream, plasma or tissue within 24, 12, 10, 8, 6, 5, 4, 3, 2, or 1 hours, or with multiple peak concentrations following administration.

In some embodiments, the critical concentration of the nicotine or a nicotine metabolite is about 1 pg/ml to about 1 mg/ml. In some embodiments the critical concentration nicotine or nicotine metabolite is about 1 pg/ml to about 1 ng/ml, or about 50 pg/ml to about 1 ng/ml, or about 100 pg/ml to about 1 ng/ml, or about 500 pg/ml to about 1 ng/ml, or about 1 ng/ml to about 500 ng/ml, or about 10 ng/ml to about 500 ng/ml, or about 100 ng/ml to about 500 ng/ml, or about 200 ng/ml to about 500 ng/ml, or about 300 ng/ml to about 500 ng/ml, or about 400 ng/ml to about 500 ng/ml, or about 500 ng/ml to about 1 ug/ml, or about 600 ng/ml to about 1 ug/ml, or about 700 ng/ml to about 1 ug/ml, or about 800 ng/ml to about 1 ug/ml, or about 900 ng/ml to about 1 ug/ml, or about 1 ug/ml to about 1 mg/ml, or about 10 ug/ml to about 1 mg/ml, or about 100 ug/ml to about 1 mg/ml, or about 500 ug/ml to about 1 mg/ml, or about 600 ug/ml to about 1 mg/ml, or about 700 ug/ml to about 1 mg/ml, or about 800 ug/ml to about 1 mg/ml, or about 900 ug/ml to about 1 mg/ml. In some embodiments, the critical concentration of the nicotine or a nicotine metabolite is about 200 ng/ml to about 420 ng/ml. In some embodiments, the critical concentration of the nicotine or a nicotine metabolite is about 1 ng/ml to about 20 ng/ml. In some embodiments, the critical concentration of the nicotine or a nicotine metabolite is about 1 ng/ml to about 5 ng/ml. In some embodiments, the critical concentration of the nicotine or a nicotine metabolite is about 20 ng/ml to about 100 ng/ml. In some embodiments, the nicotine metabolite is cotinine.

Administration of nicotine of the invention may continue as long as necessary. In some embodiments, nicotine of the invention is administered for more than 1, 2, 3, 4, 5, 6, 7, 14, 28 days or 1 year. In some embodiments, nicotine of the invention is administered chronically on an ongoing basis, e.g., for the treatment of chronic effects.

### EXAMPLES

### Example 1: Comparative Formulation

Tablets are manufactured using a dry blend process, and hand made on a Carver Press (Fred Carver, Inc., Indiana). The dry blend process consists of blending all of the ingredients, and compressing the blend into a 500 mg tablet using a standard die.

Tablets include nicotine hydrogen tartrate (nicotine bitartrate dihydrate), PEO Coagulant, Methocel K100M, and magnesium stearate. (PEO Coagulant=poly(ethylene oxide), grade PolyOx Coagulant, NF FP grade, manufactured by Union Carbide/Dow Chemical Company; Methocel K100M=hydroxypropylmethylcellulose, grade Methocel K100M, premium, manufactured by Dow Chemical Company; magnesium stearate, NF, supplied by Spectrum Chemical Company). Amounts of PEO Coagulant range from 10 to 90% by weight, amounts of Methocel K100M range from 10 to 90% by weight, and amounts of magnesium stearate range from 0 to 2% by weight.

Tablet dissolution rates are determined in USP apparatus I (40 mesh baskets), 100 rpm, in deionized water. Samples, 5 ml at each time-point, are taken without media replacement at 1, 4 and 8 hours.

### Example 2: Comparative Formulation

Example 1 is repeated with the percentage by weight of inactives as (i) 50% PEO Coagulant, 49% Methocel K100M, and 1% magnesium stearate; (ii) 89% PEO Coagulant, 10% Methocel K100M, and 1% magnesium stearate; and (iii) 10% PEO Coagulant, 89% Methocel K100M, and 1% magnesium stearate.

### Example 3: Formulation

A pulsatile release formulation is prepared with four distinct release points. Sugar spheres are coated with nicotine bitartrate dihydrate, separated into three batches, and each batch coated with enteric polymer (Eudragit L30D) in different thickness. Coated spheres are combined in equal amounts and placed in a capsule with an immediate release coating containing nicotine bitartrate dihydrate. Coating thickness for the coated spheres is altered to provide for timed pulsatile release at approximately 6, 12, and 18 hours.

Capsule dissolution rates are determined in USP apparatus I (40 mesh baskets), 100 rpm, in deionized water. Alternatively, gastric/intestinal substitute is used for dissolution studies to mimic passage through the gastrointestinal system including change in pH over time. Samples, 5 ml at each time-point, are taken without media replacement at hourly intervals.

### Example 4: Formulation

A pulsatile release coated tablet formulation is prepared with four distinct release points. Non-pareil seed cores are coated with alternating layers of nicotine bitartrate dihydrate, bioerodible coating, and enteric coating, to provide for three separate layers of nicotine bitartrate dihydrate with an outer layer of enteric coating. A final coating of nicotine bitartrate dihydrate in an immediate release layer is provided over the outer layer of enteric coating. Coating thicknesses for each layer are altered to provide for immediate release followed by additional timed pulsatile releases at approximately 6, 12, and 18 hours.

Coated tablet dissolution rates are determined in USP apparatus I (40 mesh baskets), 100 rpm, in deionized water. Alternatively, gastric/intestinal substitute is used for dissolution studies to mimic passage through the gastrointestinal system including change in pH over time. Samples, 5 ml at each time-point, are taken without media replacement at hourly intervals.

### Example 5: Clinical Trial

Patients with idiopathic PD and LIDS are enrolled in a clinical study. Major entry criteria are as follows: 1) Hoehn and Yahr Stage II-IV while in “on” state; 2) moderately to severely disabling LIDS >25% of waking day as determined by a rating of >2 on each of Questions 32 and 33 of the Unified Parkinson's Disease Rating Scale (UPDRS) or comparable measure with Unified Dyskiensia Rating Scale (UDysRS) total scores and subscores; 3) treatment with stable doses of levodopa and other medicines for PD for >30 days; 4) a negative screening urine test for cotinine; and 5) non-smoker and no regular exposure to second-hand smoke. The patient population in this study is typical of those patients with LIDS. Upon randomization of patients for receiving study agent or placebo, there are no statistically significant differences in baseline parameters between the 2 groups.

The study consists of 3 phases: a treatment period, a drug taper period, and a follow-up period. Subjects are randomly assigned to receive either a nicotine salt or placebo (pbo). Treatment is with one oral dosage per day containing a nicotine salt or placebo. For non-placebo oral dosage, dosing begins at 4 mg/day (with a release profile of 1 mg q6 hr) and is escalated at 2-week intervals to 24 mg/day (with a release profile of 6 mg q6 hr). All subjects are allowed to take rescue medication for the treatment of nausea and/or vomiting for the first 3 days of each dose escalation. Subjects are maintained on one oral dosage per day containing 24 mg/day (with a release profile of 6 mg q6 hr) for 4 weeks. Goal for medication compliance in each group is >90%.

Safety is assessed by incidences of adverse experiences (AE), clinical laboratory tests, serum cotinine, ECG and vital signs. Impulsive symptoms are assessed using the Jay Modified Minnesota Impulsive Disorders Interview (JayMidi). Withdrawal symptoms are evaluated using the Minnesota Nicotine Withdrawal Scale (MNWS-R).

Efficacy is assessed using the UPDRS (total of Parts II+III+IV), sum of Q32+Q33, Unified Dyskiensia Rating Scale (UDysRS) total scores and subscores, Lang-Fahn Dyskinesia Acitivties of Daily Living Scale (LF-ADL), CGI-C and PGI-C scales, and responder analyses (subjects with >25% improvement from baseline) on UDysRS total score and LF-ADL, and % subjects with any improvement on PGI-C and CGI-C. Multiple instruments are to be used in an exploratory manner to assess the efficacy of nicotine on LIDS. Efficacy assessment is powered to determine that treatment is generally safe and well-tolerated in PD patients with LIDS. Based on the mechanism of action of nicotine, no unexpected AEs occur. Efficacy assessment is powered to determine whether or not nicotine improves UPDRS total scores. There is a trend towards statistically significant improvement in the nicotine group compared to pbo on the majority of patient- and physician-rated outcome measures.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An oral pulsatile release dosage form for once-daily oral administration, said form comprising an effective amount of nicotine or a salt thereof for treatment of symptoms of Parkinson's Disease or symptoms associated with dopaminergic treatment of Parkinson's Disease or for delay in progression of Parkinson's Disease, wherein said form exhibits first, second, third, and fourth pulsatile releases of nicotine or salt thereof;
wherein said first pulsatile release occurs with a maximum release within two hours of administration, said second pulsatile release occurs with a maximum release between five and seven hours following administration, said third pulsatile release occurs with a maximum release between eleven and thirteen hours following administration, and said fourth pulsatile release occurs with a maximum release between sixteen and twenty hours following administration; or wherein said first pulsatile release occurs with a maximum release between about two to four hours of administration, said second pulsatile release occurs with a maximum release between seven and nine hours following administration, said third pulsatile release occurs with a maximum release between thirteen and fifteen hours following administration, and said fourth pulsatile release occurs with a maximum release between eighteen and twenty-two hours following administration; or
wherein said first pulsatile release occurs with a maximum release within two hours of entering the small intestine, said second pulsatile release occurs with a maximum release between seven and nine hours following administration, said third pulsatile release occurs with a maximum release between thirteen and fifteen hours following administration, and said fourth pulsatile release occurs with a maximum release between eighteen and twenty-two hours following administration.

2. The dosage form according to claim 1, wherein said first pulsatile release occurs with a maximum release within one hour of administration, said second pulsatile release occurs with a maximum release at about six hours following administration, said third pulsatile release occurs with a maximum release at about twelve hours following administration, and said fourth pulsatile release occurs with a maximum release at about eighteen hours following administration.

3. The dosage form according to claim 1, wherein for each pulsatile release, at least 80% of the total release of nicotine or salt thereof during said pulsatile release occurs within one hour of the time of maximum release for said pulsatile release.

4. (canceled)

5. The dosage form according to claim 3, wherein the amount is nicotine or salt thereof released outside of one hour from the time of the four pulsatile releases is less than 10% of the total amount of nicotine or salt thereof originally present in the dosage form.

6. The dosage form according to claim 1, wherein each pulsatile release comprises from about 0.1 to 10 mg nicotine or salt thereof for a total amount of nicotine or salt thereof from about 0.4 to 40 mg.

7. The dosage form according to claim 1, wherein each pulsatile release independently comprises an amount of nicotine or salt thereof selected from about 1 mg, about 2 mg, about 3 mg, about 4 mg, about 5 mg, and about 6 mg.

8. The dosage form according to claim 1, wherein each pulsatile release comprises an amount of nicotine or salt thereof selected from about 1 mg for a combined total amount in the dosage form of about 4 mg, about 2 mg for a combined total amount in the dosage form of about 8 mg, about 3 mg for a combined total amount in the dosage form of about 12 mg, about 4 mg for a combined total amount in the dosage form of about 16 mg, about 5 mg for a combined total amount in the dosage form of about 20 mg, and about 6 mg for a combined total amount in the dosage form of about 24 mg.

9. The dosage form according to claim 1, wherein said dosage comprises an effective amount of nicotine bitartrate dihydrate.

10. The dosage form according to claim 1, wherein said dosage form is a capsule.

11. The dosage form according to claim 10, wherein said capsule comprises a liquid, powder, polymeric matrix, or coating layer comprising nicotine or salt thereof for providing said first pulsatile release upon administration to a patient; and said capsule further comprises beads comprising nicotine or salt thereof for providing said second, third, and fourth pulsatile releases upon administration to a patient.

12. The dosage form according to claim 11, wherein said beads are selected from the group consisting of enteric-coated beads, erodible-matrix beads, wax-coated beads, ethylcellulose-coated beads, silicone elastomer coated beads, and combinations thereof.

13. The dosage form according to claim 10, wherein said capsule comprises a water-swellable polymeric membrane, and wherein said water-swellable polymeric membrane ruptures following administration to a patient.

14. (canceled)

15. The dosage form according to claim 10, wherein said capsule comprises a hard gelatin outer surface.

16. The dosage form according to claim 1, wherein said dosage form is a tablet.

17. The dosage form according to claim 16, wherein said tablet comprises an immediate release coating and a core, wherein said immediate release coating comprises nicotine or salt thereof for said first pulsatile release, and said core comprises nicotine or salt thereof for said second, third, and fourth pulsatile releases, and wherein said immediate release coating is selected from an erodible-matrix coating, a wax coating, an ethylcellulose coating, a silicone elastomer coating, and combinations thereof.

18. (canceled)

19. The dosage form according to claim 1, wherein said dosage form further comprises levodopa, carbidopa, or a combination thereof.

20. A method for treatment of symptoms of Parkinson's Disease or symptoms associated with dopaminergic treatment of Parkinson's Disease or for delay in progression of Parkinson's Disease, said method comprising administering a dosage form according to claim 1.

21. The method of claim 20, wherein said symptoms of Parkinson's Disease are gait and balance problems.

22. The method of claim 20, wherein said symptoms associated with dopaminergic treatment of Parkinson's Disease are levodopa-induced dyskinesias.

23. The method of claim 20, wherein said dosage form is capable of being administered so that one or more metabolites of said nicotine or salt thereof achieves a plasma level of about 1 to about 500 ng/ml within one hour of each of said pulsatile releases.
